(19) United States
(12) Patent Application Publication
(10) Pub. No.: US 2007/0142412 A1
(43) Pub. Date: Jun. 21, 2007

(54) Crystalline form of 2-{4-['3-(4-chloro-2-fluorophenyl)-4-pyrimidin-4-yl-1h-pyrazol-5-yl]piperidin-1-yl}-2-oxoethanol

(76) Inventors: Richard R. Schartman, Wallingford, CT (US); Douglas H. Hoffman, Moorpark, CA (US)

Correspondence Address: PHARMACIA & UPJOHN, 7000 Portage Road, KZO-300-104, KALAMAZOO, MI, 49001, US

(21) Appl. No.: 10/583,460

(22) PCT Filed: Dec. 15, 2004
(86) PCT No.: PCT/IB04/04187

§ 371 Date: Sep. 22, 2006

Related U.S. Application Data
(60) Provisional application No. 60/530,763, filed on Dec. 19, 2003.

(51) Int. Cl. A61K 31/506; C07D 403/14

(52) U.S. Cl. 514/275; 544/331

(57) ABSTRACT

Crystalline form of the p38 kinase inhibitor 2-{4-[3-(4-chloro-2-fluorophenyl)-4-pyrimidin-4-yl-1H-pyra-zol-5-yl]piperidin-1-yl}-2-oxoethanol can be used, is provided. The crystalline form is a hydrated crystalline form. Also provided are combinations and pharmaceutical compositions comprising the crystalline form, process for preparing the crystalline form and for preparing compositions comprising the crystalline form, in methods for the prophylaxis and/or treatment of a p38 kinase-mediated condition comprising administering to a subject a therapeutically effective amount of the crystalline form of 1.

This application claims priority to U.S. Provisional application number 60/530,763 filed Dec. 19, 2003.

### FIELD OF THE INVENTION

This invention is in the field of pharmaceutical agents active as p38 kinase inhibitors, and more particularly concerns the p38 kinase inhibitor 2-{4-[3-(4-chloro-2-fluorophenyl)-4-pyrimidin-4-yl-1H-pyrazol-5-yl]piperidin-1-yl}-2-oxoethanol. Specifically, the invention relates to a novel hydrate form of2-{4-[3-(4-chloro-2-fluorophenyl)-4-pyrimidin-4-yl-1H-pyrazol-5-yl]piperidin-1-yl}-2-oxoethanol.

### BACKGROUND OF THE INVENTION

The compound 2-{4-[3-(4-chloro-2-fluorophenyl)-4-pyrimidin-4-yl-1H-pyrazol-5-yl]piperidin-1-yl}-2-oxoethanol having the structure (1) below (referred to herein as “Compound 1”) is described in WO 03/104223. WO 03/104223 discloses a class of substituted pyrazole compounds and related pharmaceutical compositions that are useful for the treatment and/or prophylaxis of a p38 kinase-mediated condition, example of such include inflammation and inflammation related conditions. Example 27 of WO 03/104223_specifically discloses Compound land methods for the synthesis of Compound 1.

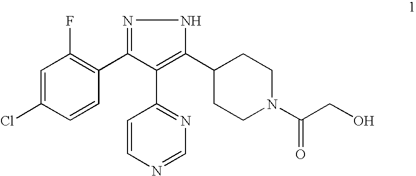

A need exists for a crystalline form of Compound 1 that is physically stable and sufficiently bioavailable, and for reliable and reproducible processes for the manufacture and/or purification of such crystalline form. There is now provided a novel crystalline form of Compound 1 having a high degree of physical stability at common temperatures of storage and use.

### SUMMARY OF THE INVENTION

The invention provides, in a first aspect, a hydrous crystalline form of Compound 1(the “Form 1 hydrate”).

In another aspect, the invention provides pharmaceutical compositions comprising the Form 1 hydrate, and further optionally comprising one or more pharmaceutically acceptable excipients.

In another aspect, the invention provides pharmaceutical compositions containing about 0.1 mg to about 1000 mg of the Form 1 hydrate.

In another aspect, the invention provides a process for preparing the Form 1 hydrate and for preparing compositions comprising the Form 1 hydrate.

In another aspect, the invention provides a method for prophylaxis and/or treatment of p38 kinase-mediated condition comprising administering to a subject a therapeutically effective amount of the Form 1 hydrate.

Additional aspects of the invention will be in part apparent and in part pointed out throughout this application.

### BRIEF DESCRIPTION OF THE DRAWINGS

FIG. 1 shows an illustrative X-ray powder diffraction pattern for the Form 1 hydrate of Compound 1.

FIG. 2 shows an illustrative differential scanning calorimetry thermogram of Form 1 hydrate of Compound 1.

FIG. 3 shows an illustrative infrared (IR) spectrum (attenuated total reflectance, ATR) of the Form 1 hydrate of Compound 1.

FIG. 4 shows an illustrated moisture sorption profile of the Form 1 hydrate.

FIG. 5 shows an illustrated moisture sorption profile of the Form 1 hydrate over the 0-30% relative humidity range.

### DETAILED DESCRIPTION OF THE INVENTION

As with other pharmaceutical compounds and compositions, the chemical and physical properties of 2-{4-[3-(4-chloro-2-fluorophenyl)-4-pyrimidin-4-yl-1H-pyrazol-5-yl]piperidin-1-yl}-2-oxoethanol (“Compound 1”) are important in its commercial development. These properties include, but are not limited to: (1) packing properties such as molar volume, density and hygroscopicity, (2) thermodynamic properties such as melting temperature, vapor pressure and solubility, (3) kinetic properties such as dissolution rate and stability (including stability at ambient conditions, especially to moisture, and under storage conditions), (4) surface properties such as surface area, wettability, interfacial tension and shape, (5) mechanical properties such as hardness, tensile strength, compactibility, handling, flow and blend, (6) filtration properties, (7) chemical purity, and (8) physical and chemical stability. These properties can affect, for example, processing and storage of pharmaceutical compositions comprising Compound 1. Solid-state forms of Compound 1 that provide an improvement in one or more of these properties relative to other solid-state forms of Compound 1 are desirable.

According to the present invention, therefore, a new solid-state form of Compound 1 has been discovered. The specific solid-state form of Compound 1 that has been discovered includes a hydrous crystalline form possessing thermodynamic stability under normal manufacturing conditions.

In one embodiment, the invention comprises the Form 1 hydrate of Compound 1. The Form 1 hydrate possesses physical stability at ambient temperatures. Solid-state forms of Compound 1 that do not require special processing or storage conditions, and that avoid the need for frequent inventory replacement, such as the Form 1 hydrate, are desirable. For example, selection of a solid-state form of Compound 1 that is physically stable during a manufacturing process (such as during milling of Compound 1 to obtain a material with reduced particle size and increased surface area) can avoid the need for special processing conditions and the increased costs generally associated with such special processing conditions. Similarly, selection of a solid-state form of Compound 1 that is physically stable over a wide range of storage conditions (especially considering the different possible storage conditions that can occur during the lifetime of a Compound 1 product) can help avoid polymorphic or other degradative changes in the Compound 1 that can lead to product loss or deterioration of product efficacy. Therefore, the selection of a solid-state form of Compound 1 such as the Form 1 hydrate having greater physical stability provides a meaningful benefit over less stable Compound 1 solid-state forms.

Indications

The solid-state form of Compound 1 described in this application is useful for, but not limited to, the treatment of any condition in a human, or other mammal, which is exacerbated or caused by excessive or unregulated cytokine production by the mammal, such as TNF or p38 kinase production. The solid-state forms of Compound 1 is p38 kinase antagonists, directly or indirectly antagonize cytokines such as TNF and IL-1 proteins, and/or have the ability to retard the natural course ofjoint destruction in rheumatoid arthritis patients. Accordingly, the present invention provides a method of treating a cytokine-mediated condition, which comprises administering to a subject an effective cytokine-interfering amount of a solid-state form of Compound 1.

The solid-state form of Compound 1 is useful for, but not limited to, the treatment or prophylaxis of:
(1) inflammation;
(2) arthritis including rheumatoid arthritis, spondyloarthropathies, gouty arthritis, osteoarthritis, systemic lupus erythematosus and juvenile arthritis, osteoarthritis, and other arthritic conditions;
(3) neuroinflammation;
(4) allergy, Th2 mediated diseases;
(5) pain (i.e., use as an analgesic) including but not limited to neuropathic pain;
(6) fever (i.e., use as an antipyretic);
(7) pulmonary disorders or lung inflammation, including adult respiratory distress syndrome, pulmonary sarcoidosis, asthma, silicosis, chronic pulmonary inflammatory disease, chronic obstructive pulmonary disease (COPD), and asthma;
(8) cardiovascular diseases including atherosclerosis, myocardial infarction (including post-myocardial infarction indications), thrombosis, congestive heart failure, and cardiac reperfusion injury, as well as complications associated with hypertension and/or heart failure such as vascular organ damage, restenosis;
(9) cardiomyopathy;
(10) stroke including ischemic and hemorrhagic stroke;
(11) ischemia including brain ischemia and ischemia resulting from cardiac/coronary bypass;
(12) reperfusion injury (13) renal reperfusion injury;
(14) brain edema;
(15) neurotrauma and brain trauma including closed head injury;
(16) neurodegenerative disorders;
(17) central nervous system disorders (including, but not limited to, central nervous system disorders having an inflammatory or apoptotic component), such as Alzheimer's disease, Parkinson's disease, Huntington's Disease, amyotrophic lateral sclerosis, spinal cord injury, and peripheral neuropathy. (18) liver disease and nephritis;
(19) gastrointestinal conditions such as inflammatory bowel disease, Crohn's disease, gastritis, irritable bowel syndrome and ulcerative colitis;
(20) ulcerative diseases such as gastric ulcer;
(21) periodontal disease (22) ophthalmic diseases such as retinitis, retinopathies (including diabetic retinopathy), uveitis, ocular photophobia, nonglaucomatous optic nerve atrophy, and age related macular degeneration (ARMD) (including ARMD-atrophic form);
(23) ophthalmological conditions such as corneal graft rejection, ocular neovascularization, retinal neovascularization including neovascularization following injury or infection, and retrolental fibroplasia;
(24) glaucoma including primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation and corticosteroid-induced glaucoma;
(25) acute injury to the eye tissue and ocular traumas such as post-traumatic glaucoma, traumatic optic neuropathy, and central retinal artery occlusion (CRAO);
(26) diabetes;
(27) diabetic nephropathy;
(28) skin-related conditions such as psoriasis, eczema, bums, dermatitis, keloid formation, scar tissue formation, and angiogenic disorders;
(29) viral and bacterial infections, including sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, and herpes virus;
(30) myalgias due to infection;
(31) influenza;
(32) endotoxic shock, sepsis;
(33) toxic shock syndrome;
(34) autoimmune disease including graft vs. host reaction and allograft rejections;
(35) treatment of bone resorption diseases, such as osteoporosis;
(36) multiple sclerosis;
(37) disorders of the female reproductive system such as endometriosis;
(38) pathological, but non-malignant, conditions such as hemaginomas, including infantile hemaginomas, angiofibroma of the nasopharynx and avascular necrosis of bone;
(39) benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer and skin cancer, such as squamus cell and basal cell cancers, prostate cancer, renal cell carcimoma, and other known cancers that affect epithelial cells throughout the body;
(40) leukemia;
(41) lymphoma;
(42) systemic lupus erthrematosis (SLE);
(43) angiogenesis including neoplasia; and
(44) metastasis.

The crystalline form of Compound 1 disclosed in this application is also useful for preventing the production or expression of cyclooxygenase-2, or cyclooxygenase-2 activity.

Definitions

The term “crystalline form” as applied to Compound 1 herein refers to a solid-state form wherein the Compound 1 molecules are arranged to form a distinguishable crystal lattice (i) comprising distinguishable unit cells, and (ii) yielding diffraction peaks when subjected to X-ray radiation.

The term “crystallization” as used herein can refer to crystallization and/or recrystallization depending upon the applicable circumstances relating to preparation of Compound 1 starting material.

The term “direct crystallization” as used herein refers to crystallization of Compound 1 directly from a suitable solvent without formation and desolvation of an intermediate solvated crystalline solid-state form of Compound 1.

The term “Compound 1 drug substance” as used herein means Compound 1 per se as qualified by the context in which the term is used, and can refer to unformulated Compound 1 or to Compound 1present as an ingredient of a pharmaceutical composition.

The term “particle size” as used herein refers to particle size as measured by conventional particle size measuring techniques well known in the art, such as laser light scattering, sedimentation field flow fractionation, photon correlation spectroscopy or disk centrifugation. One nonlimiting example of a technique that can be used to measure particle size is a liquid dispersion technique employing a Sympatec Particle Size Analyzer. The “D₉₀ particle size” is a particle size such that 90% by weight of the particles are smaller than the D₉₀ particle size as measured by such conventional particle size measuring techniques.

The term “DSC” means differential scanning calorimetry.

The term “HPLC” means high pressure liquid chromatography.

The term “IR” means infrared.

The term “msec” means millisecond.

The term “purity” herein, unless otherwise qualified, means the chemical purity of Compound 1 according to conventional HPLC assay.

The term “phase purity” herein means the solid-state purity of Compound 1with regard to a particular crystalline or amorphous form of the Compound 1 as determined by X-ray powder diffraction analytical methods described herein. The term “phase pure” refers to purity with respect to other solid-state forms of Compound 1 and does not necessarily imply a high degree of chemical purity with respect to other compounds.

The term “PXRD” means X-ray powder diffraction.

The term “TGA” means thermogravimetric analysis.

Characterization of Crystalline Form 1

1. X-Ray Diffraction

Single crystal X-ray analyses of the Form 1 hydrate of Compound 1 were conducted using a Siemens D5000 diffractometer with a theta,theta configuration, CuKα radiation, 2.0-second step time, 0.020-degree step size, and a plastic sample holder. The broad band at about 12.5 degrees Two-Theta is due to the sample holder.

(1) Table 1 presents data obtained for a sample of the Form 1 hydrate.

TABLE 1
X-Ray Diffraction Data

| Angle (2-theta degrees) | d-value | Intensity (Counts) | Intensity (%) |
|---|---|---|---|
| 8.346 | 10.58565 | 1367 | 24.2 |
| 10.595 | 8.34301 | 416 | 7.4 |
| 11.773 | 7.51094 | 2217 | 39.3 |
| 12.709 | 6.95947 | 907 | 16.1 |
| 14.016 | 6.31353 | 960 | 17 |
| 15.084 | 5.86883 | 243 | 4.3 |
| 15.553 | 5.69279 | 270 | 4.8 |
| 16.702 | 5.30362 | 2453 | 43.5 |
| 17.172 | 5.15937 | 879 | 15.6 |
| 17.381 | 5.09803 | 1143 | 20.3 |
| 17.853 | 4.96415 | 3008 | 53.3 |
| 19.678 | 4.50767 | 405 | 7.2 |
| 19.836 | 4.47221 | 460 | 8.2 |
| 20.76 | 4.27516 | 833 | 14.8 |
| 21.215 | 4.18449 | 5642 | 100 |
| 21.858 | 4.06275 | 1142 | 20.2 |
| 22.16 | 4.00807 | 1367 | 24.2 |
| 22.846 | 3.88927 | 676 | 12 |
| 23.513 | 3.78045 | 1417 | 25.1 |
| 23.74 | 3.74483 | 779 | 13.8 |
| 24.857 | 3.57908 | 2109 | 37.4 |
| 25.119 | 3.54234 | 1138 | 20.2 |
| 26.251 | 3.3921 | 490 | 8.7 |
| 26.913 | 3.31008 | 226 | 4 |
| 27.725 | 3.21496 | 2089 | 37 |
| 28.15 | 3.16734 | 1065 | 18.9 |
| 28.556 | 3.1233 | 1334 | 23.6 |
| 29.762 | 2.9994 | 864 | 15.3 |
| 30.393 | 2.93857 | 494 | 8.8 |
| 31.17 | 2.86707 | 849 | 15 |
| 32.308 | 2.76863 | 634 | 11.2 |
| 33.281 | 2.68981 | 493 | 8.7 |
| 33.595 | 2.6654 | 316 | 5.6 |
| 35.084 | 2.5556 | 217 | 3.8 |
| 35.745 | 2.50988 | 376 | 6.7 |
| 36.088 | 2.48678 | 614 | 10.9 |
| 37.458 | 2.39895 | 275 | 4.9 |
| 38.14 | 2.35761 | 576 | 10.2 |
| 39.466 | 2.28137 | 281 | 5 |
| 40.329 | 2.23453 | 343 | 6.1 |
| 40.824 | 2.20858 | 266 | 4.7 |
| 41.479 | 2.17522 | 213 | 3.8 |
| 42.46 | 2.1272 | 295 | 5.2 |
| 42.885 | 2.10709 | 218 | 3.9 |
| 43.226 | 2.09125 | 195 | 3.5 |
| 43.884 | 2.06138 | 278 | 4.9 |
| 44.417 | 2.0379 | 247 | 4.4 |
| 45.292 | 2.00052 | 306 | 5.4 |
| 45.832 | 1.97823 | 346 | 6.1 |
| 46.698 | 1.94354 | 250 | 4.4 |
| 47.623 | 1.90791 | 212 | 0.8 |
| 48.071 | 1.89117 | 296 | 5.2 |
| 48.525 | 1.87454 | 247 | 4.4 |
| 49.447 | 1.84172 | 247 | 4.4 |

The Form 1 hydrate typically has an X-ray powder diffraction pattern comprising at least one peak selected from the group consisting of 8.3±0.2, 11.7 ±0.2, 16.7±0.2, 21.2±0.2, 24.8+0.2, 27.7±0.2, and 28.5±0.2 degrees 2 theta. In one embodiment of the invention, the solid-state form of Compound 1 is the Form 1 hydrate having an X-ray powder diffraction pattern comprising peaks at 11.7±0.2 and 28.5 ±0.2 degrees 2 theta.

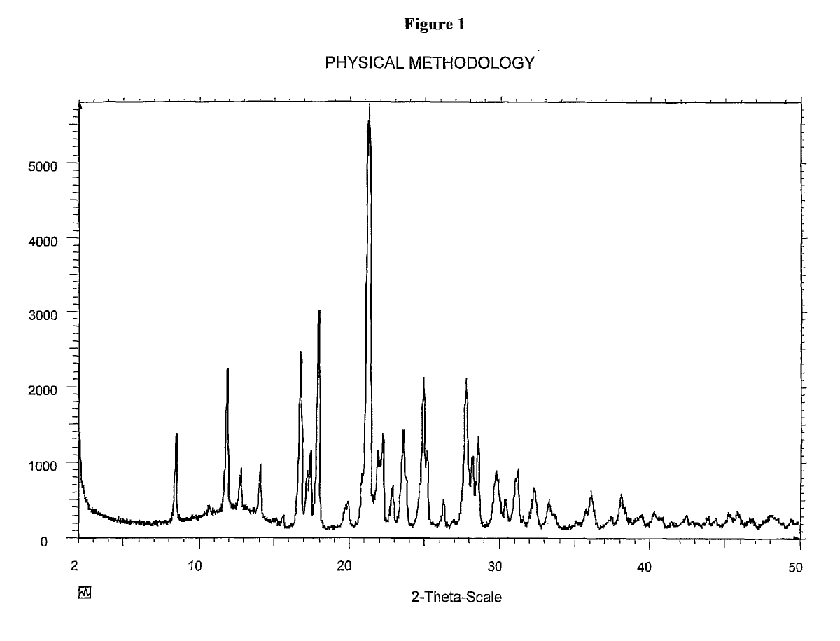

FIG. 1 shows an illustrative X-ray powder diffraction pattern for the Form 1 hydrate of Compound 1.

2. Differential Scanning Calorimetry (DSC)

DSC data of the hydrated form of Compound 1 were determined using a TA Instruments 2920 differential scanning calorimeter. Each sample (an amount of about 1 mg to about 2 mg) was placed in an unsealed aluminum pan and heated at 10° C./minute, and nitrogen purge. Transition temperature ranges were defined from the extrapolated onset to the maximum of the peak.

Table 2 below summarizes typical DSC measurements obtained for the crystalline form of Compound 1.

TABLE 2
DSC Analysis

| | | Temperature |
|---|---|---|
| Crystalline Form | Thermal Event | ° C. |
| Form 1 hydrate | (a) Exothermic (crystallization) | 150-158 |
| | (b) Endothermic (melt and degradation) | 213-217 |
| | (c) Exothermic (degradation) | 219-222 |

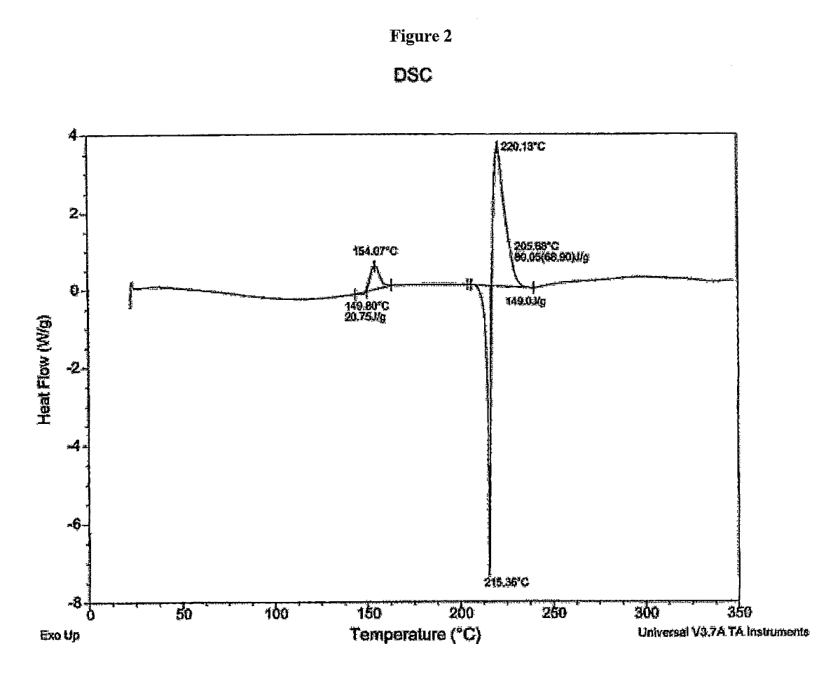

FIG. 2 shows an illustrative differential scanning calorimetry thermogram of Form 1 hydrate of Compound 1.

3. Thermogravimetric Analysis

Thenrogravimetric analysis of Form 1 was performed using a TA Instruments TGA Q500 theromgravimetric analyzer. Samples were placed in an unsealed aluminum pan under nitrogen purge. Data was collected from room temperature to 350° C. at 10° C./minute. The table below summarizes typical thermogravimetry measurements obtained for Form 1.

TABLE 3
Thermogravimetric Analysis (TGA)

| | | Temperature | Weight Loss |
|---|---|---|---|
| Crystalline Form | Thermal Event | ° C. | (%) |
| Form 1 hydrate | Loss of approximately | 30-150° C. | 5.7% |
| | 1.5 moles of water. | | |

4. Infrared Spectroscopy

The ATR-IR data were obtained using neat chemical, a SensIR Duroscope micro diamond ATR accessory, and a Digilab Model FTS-45 spectrometer. No pressure was applied to the sample.

TABLE 4
IR Bands (cm⁻¹)

| Frequency (cm⁻¹) | Assignments^{b} |
|---|---|
| 3391 (broad) | ν OH & ν NH |
| 3100-3000 | ν ═CH (aromatic) |
| 1644 | ν C═O |
| 1586 (broad), 1502 | ν C═C (aromatic), pyrazole & pyrimidine ring |
| (broad) | stretching modes (ν C═C, C═N) |
| 1442 (broad) | ν C═C (aromatic), pyrazole & pyrimidine ring |
| | stretching modes (ν C═C, C═N), δ CH₂(CCH₂ & |
| | NCH₂) |
| 1392, 1374 (weak) | pyrazole ring stretching mode & δ OH |
| 1222 | ν ═C—F, pyrimidine ═CH |
| 1093 | ν ═C—C1 |
| 994*, 976 | pyrazole & pyrimidine ring breathing modes, |
| | *also ν C—O (p-alcohol) |
| 890, 862/854, 834 | δ ═CH (isolated and 2 adjacent H's on benzene and |
| | 2-substituted pyrimidine) |

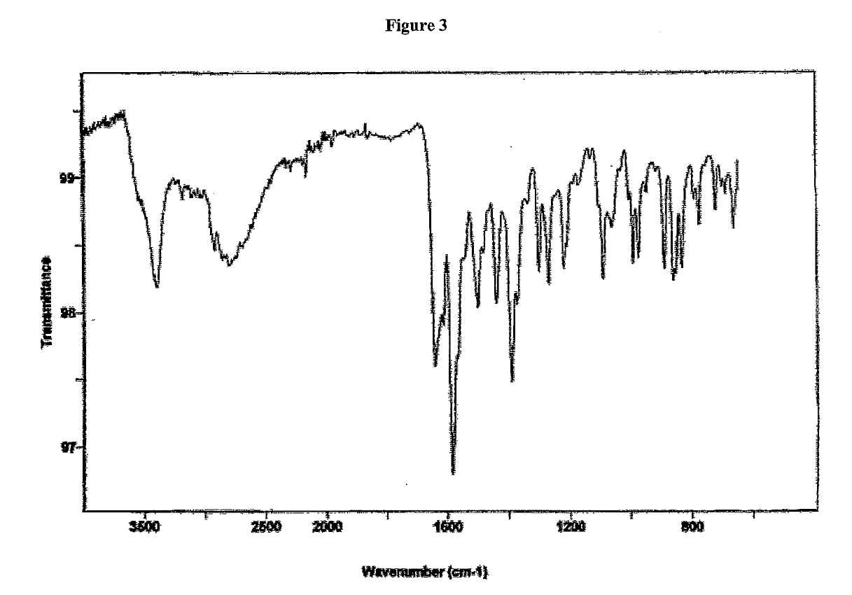

FIG. 3 shows an illustrative infrared (IR) spectrum (attenuated total reflectance, ATR) of the Form 1 hydrate of Compound 1.

5. Unit Cell Parameters

A supersaturated solution of the Form 1 hydrate in ethanol was produced at approximately 6 mg/mL. The sample was heated to approximately 60° C. using a Pierce Reacti-Therm to dissolve the solid. The resulting solution was then transferred to an HPLC vial. The HPLC vial was then placed inside a scintillation vial containing HPLC water. The cap to the scintillation vial was only loosely tightened. The sample was maintained at room temperature for approximately three weeks at which time single crystals were observed.

The single crystal X-ray data for the Form 1 hydrate, were collected using CuKα radiation and a SMART 6K CCD X-ray area detector with window diameter =13.5 cm.

Table 5 below summarizes the unit cell parameters determined for the Form 1 hydrate.

TABLE 5
Unit Cell Parameters

| Parameter | Form 1 hydrate |
|---|---|
| Crystal System | Monoclinical |
| Empirical | C₂₀H₁₉ClFN₅O₂•1.5H₂O |
| Formula | |
| Formula Weight | 442.86 |
| a (Å) | 19.5924(4) |
| b (Å) | 13.8492(3) |
| c (Å) | 17.7953(4) |
| beta (⁰) | 122.3660(10) |
| density | 1.44 g/cm³ |
| Z | 8 |
| Space group | C2/c |

6. Moisture Sorption Analysis

The moisture sorption profile of the sample was determined using a Surface Measurement System (SMS) DVS-1 Automated Water Sorption Analyzer operating via SMS Software version 2.16. The change in mass of the sample versus relative humidity (RH) was monitored at 25° C. using a method from 30% to 0%, 0% to 90%, 90% to 0%, and 0% to 30% RH in 10% RH steps with dm/dt=3×10⁻⁴. Maximum hold time per step was 4 hours. An approximately 15 mg sample was loaded onto the sample holder. The balance was calibrated with a 100 mg standard weight at 25° C. HPLC-grade water was used for the study.

It is believed that about 0.5% water is believed to be surface bound moisture. To investigate the hydration state of the Form 1 hydrate, moisture sorption analysis was performed. At the conclusion of the study, the sample was removed from the moisture sorption balance and analyzed by PXRD. No change was observed in the PXRD diffraction pattern of the material after moisture sorption analysis. Constant mass was not obtained at 0% RH in the moisture sorption study; therefore, an additional moisture sorption study was conducted with increase maximum hold time per step to allow the sample to reach equilibrium over the low RH range. The moisture sorption data indicate that the Form 1 hydrate contains approximately 5.5% water, which at least 0.5% is thought to be surface bound moisture. This theory is in no way to be construed as limiting.

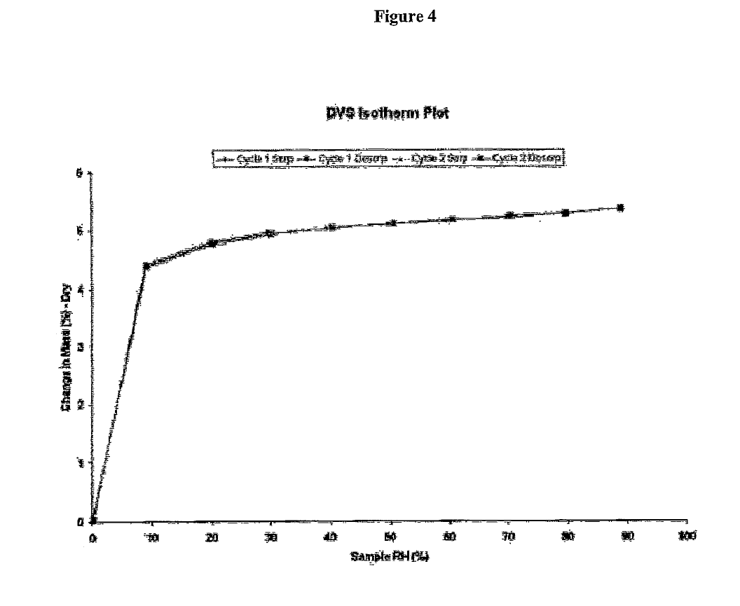

FIG. 4 shows an illustrated moisture sorption profile of the Form 1 hydrate over the 0%-90% relative humidity range.

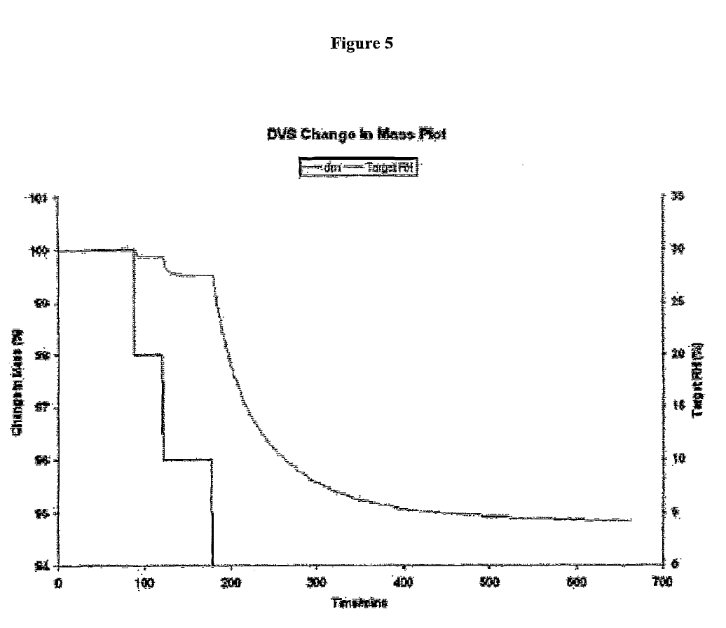

FIG. 5 shows an illustrated moisture sorption profile of the Form 1 hydrate over the 0%-30% relative humidity range.

7. Coulometric Karl Fischer Titration (KF)

The water content of samples was measured using a Mettler DL37 KF Coulometer. The background water content was determined by simulating loading a sample into the titrator. The sample was accurately weighed and quickly transferred to the titrator before measurement. The amount of water titrated for the blank was subtracted from that obtained for the sample. The percentage of water, expressed as percentage w:w, for the sample was then calculated using the corrected water content.

Table 6 shows the elemental analysis, TGA and KF titrimetry data for the Form 1 hydrate. Also shown in table 6 are theoretical values for a monohydrate and a sesquihydrate of Compound 1. As indicated by table 6, a monohydrate of Compound 1 would theoretically contain 4.15% water by weight. The elemental analysis, TGA and KF titrimetry indicate that the Form 1 hydrate contains approximately 5.9% water; however, this amount of water is more typical of a sesquihydrate of Compound 1. Elemental analysis, TGA and KF titrimetry, however do not distinguish between surface bound moisture and water in the crystal lattice.

| | | Theory (%) in | Theory (%) in |
|---|---|---|---|
| | | monohydrate | sesquihydrate |
| | Found (%) | of Compound 1 | of Compound 1 |
| C | 54.12 | 55.37 | 54.24 |
| H | 5.23 | 4.88 | 5.01 |
| N | 15.78 | 16.14 | 15.81 |
| Cl | Not | 8.17 | 8.01 |
| | determined | | |
| Water content by | 5.93 | 4.15 | 6.10 |
| KF | | | |
| Mass loss by | 5.90 | 4.15 | 6.10 |
| TGA | | | |

It is believed that the Form 1 hydrate can exist in various hydrate forms. In one embodiment the crystalline structure of the Form 1 hydrate can comprise about 1 mol water per mol of Compound 1. In another embodiment the crystalline structure of the Form 1 hydrate can comprise about 1.25 mol water per mol of Compound 1. In another embodiment the crystalline structure of the Form 1 hydrate can comprise about 1.5 mol water per mol of Compound 1. In another embodiment the crystalline structure of the Form 1 hydrate can comprise a range between about 1 mol to about 1.5 mol water per mol of Compound 1.

Pharmaceutical Compositions

The present invention is further directed to pharmaceutical compositions comprising the crystalline form of Compound 1. In one embodiment, the pharmaceutical composition comprises the Form 1 hydrate and (ii) one or more pharmaceutically acceptable carriers and/or diluents and/or adjuvants (collectively referred to herein as “excipients”) and, optionally, (iii) one or more active ingredients other than Compound 1.

In another embodiment, essentially the entire amount of Compound 1 contained in the composition is present as substantially phase pure Form 1 hydrate.

In one embodiment, at least a detectable fraction of Compound 1 is present in the form of the Form 1 hydrate.

In another embodiment, at least fifty percent (50%) of Compound 1 is present in the form of the Form 1 hydrate.

In another embodiment, at least ninety percent (90%) of Compound 1 is present in the form of the Form 1 hydrate.

The compound of the present invention can be administered to the subject as the neat compound alone. Alternatively the compounds of the present invention can be presented with one or more pharmaceutically acceptable excipients in the form of a pharmaceutical composition. A useful excipient can be, for example, a carrier. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well known techniques of pharmacy, consisting essentially of admixing the components.

These compounds can be administered by any conventional means available for use in conjunction with pharmaceuticals, either as individual therapeutic compounds or as a combination of therapeutic compounds.

The amount of compound which is required to achieve the desired biological effect will, of course, depend on a number of factors such as the specific compound chosen, the use for which it is intended, the mode of administration, and the clinical condition of the recipient.

The compositions of the invention generally can be presented in a dosage form containing about 0.1 mg to about 1000 mg of the crystalline form of Compound 1. In other embodiments, the dosage form contains about 0.1 mg to about 500 mg, 0.2 mg to about 600 mg, about 0.3 mg to about 250 mg, about 0.4 mg to about 150 mg, about 0.5 mg to about 100 mg, about lmg to about 100 mg, about 0.6 mg to about 50 mg, about 0.7 mg to about 25 mg, about 0.8 mg to about 15 mg, about 0.9 mg to about 10 mg, or about 1 mg to about 5 mg of the crystalline form of Compound 1. In still other embodiments, the dosage form contains less than about 100 mg, less than about 75 mg, less than about 50 mg, less than about 25 mg, or less than about 10 mg of the crystalline form of Compound 1. This total daily dose can be administered to the patient in a single dose, or in proportionate multiple subdoses. Subdoses can be administered 2 to 6 times per day. Doses can be in sustained release form effective to obtain desired results.

Illustrative non-limiting dosage unit forms of the pharmaceutical compositions can typically contain, for example, 0.1, 0.2, 0.5, 1, 2, 3, 4, 5, 6, 7, 8, 9 10, 20, 25, 30, 37.5, 40, 50, 75, 100, 125, 150, 175, 200, 250, 300, 350 or 400 mg ofthe crystalline form of Compound 1.

Oral delivery of the compound of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric- coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

When administered intravenously, the daily dose can, for example, be in the range of from about 0.1 mg/kg body weight to about 20 mg/kg body weight, preferably from about 0.25 mg/kg body weight to about 10 mg/kg body weight, more preferably from about 0.4 mg/kg body weight to about 5 mg/kg body weight. This dose can be conveniently administered as an infusion of from about 10 ng/kg body weight to about 2000 ng/kg body weight per minute. Infusion fluids suitable for this purpose can contain, for example, from about 0.1 ng to about 10 mg, preferably from about 1 ng to about 200 mg per milliliter. Unit doses can contain, for example, from about 1 mg to about 200 g of the compound of the present invention. Thus, ampoules for injection can contain, for example, from about 1 mg to about 200 mg.

Pharmaceutical compositions according to the present invention include those suitable for oral, rectal, topical, buccal (e.g., sublingual), and parenteral (e.g., subcutaneous, intramuscular, intradennal, or intravenous) administration, although the most suitable route in any given case will depend on the nature and severity of the condition being treated and on the nature of the particular compound which is being used. In most cases, the preferred route of administration is oral.

Formulations suitable for topical administration to the eye also include eye drops wherein the active ingredients are dissolved or suspended in suitable carrier, especially an aqueous solvent for the active ingredients. The anti-inflammatory active ingredients are preferably present in such formulations in a concentration of 0.5 to 20%, advantageously 0.5 to 10% and particularly about 1.5% w/w.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection. Such preparations can conveniently be prepared by admixing the compound with water and rendering the resulting solution sterile and isotonic with the blood. Injectable compositions according to the invention will generally contain from 0.1 to 5% w/w of a compound disclosed herein.

Pharmaceutical compositions suitable for rectal administration are preferably presented as unit-dose suppositories. These can be prepared by admixing a compound of the present invention with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which can be used include vaseline, lanoline, polyethylene glycols, alcohols, and combinations of two or more thereof. The active compound is generally present at a concentration of from 0.1 to 15% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound of the present invention in an optionally buffered, aqueous solution, dissolved and/or dispersed in an adhesive, or dispersed in a polymer. A suitable concentration of the active compound is about 1% to 35%, preferably about 3% to 15%. As one particular possibility, the compound can be delivered from the patch by electrotransport or iontophoresis, for example, as described in Pharmaceutical Research, 3(6), 318 (1986).

In any case, the amount of active ingredient that can be combined with carrier materials to produce a single dosage form to be administered will vary depending upon the host treated and the particular mode of administration.

The solid dosage forms for oral administration including capsules, tablets, pills, powders, and granules noted above comprise one or more compounds of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Injectable preparations, for example, sterile injectable aqueous or oleaginous suspensions may be fonnulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutically acceptable carriers encompass all the foregoing and the like.

Methods of Treatment and/or Prophylaxis

The present invention also embraces a method for treatment and/or prophylaxis of a p38 kinase-mediated condition, the method comprising treating a subject having or susceptible to such condition or disorder with a therapeutically effective amount of a solid-state form of Compound 1 or a pharmaceutical composition containing a solid-state form of Compound 1.

In one embodiment the p38 kinase-mediated condition is rheumatoid arthritis.

Such a method is useful for treatment and/or prophylaxis of a condition in a subject where administration of a p38 kinase inhibitor is indicated, including, but not limited to, treatment of those conditions previously disclosed above.

Besides being useful for human treatment, the solid-state forms of Compound 1 and pharmaceutical compositions thereof are also useful for veterinary treatment of companion, exotic and farm animals, for example horses, dogs, and cats.

The solid-state forms of Compound 1 and compositions thereof also can be used (i) in therapies partially or completely in place of other anti-inflammatory drugs, and/or (ii) in combination therapies with other drugs. Such anti-inflammatory and other drugs may include, but are not limited to, steroids, cyclooxygenase-2 inhibitors, DMARD'S, immunosuppressive agents, NSAIDs, 5-lipoxygenase inhibitors, LTB₄ antagonists and LTA₄ hydrolase inhibitors. The plirase “combination therapy” embraces administration of each drug in a sequential manner in a regimen that will provide beneficial effects of the drug combination, as well as co-administration of the drugs in a substantially simultaneous maimer, such as in a single capsule or injection having a fixed ratio of these active agents or in multiple, separate dosage forms or injections, one for each agent.

### EXAMPLES

The following Examples contain detailed descriptions of methods of preparation of the crystalline form of Compound 1 described herein. This detailed descriptions fall within the scope of the invention and illustrate the invention without in any way restricting that scope. All percentages are by weight unless otherwise indicated.

### Example 1.

Preparation of the Benzoyl Chloride:

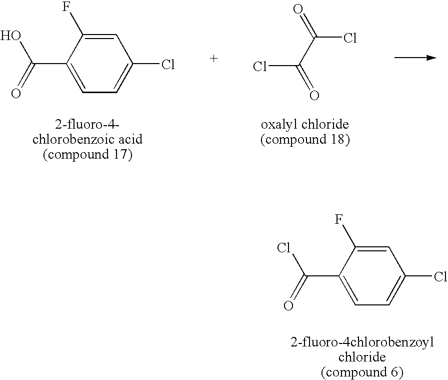

A 12L round bottom flask was equipped with a large diameter gas outlet tube, 1L addition funnel, nitrogen sweep, and overhead stirrer. To this vessel was charged 1360 g (7.79 moles, 1 equivalent) of 2-fluoro-4-chlorobenzoic acid. This was followed by addition of 5.0 liters of dry tetrahydrofuran (THF), which readily dissolved the white fluffy solid to give a yellowish clear solution. To this stirring solution was added 13.6 g of dimethylformamide (DMF). Oxalyl chloride (1088 g, 8.57 moles, 1.1 equivalent) placed in the addition funnel was added dropwise. As the addition progresses, the batch temperature increased to ca. 38° C. Afterwards the batch was heated to 42° C. and held until no remaining starting material was left. The batch was cooled to room temperature and a nitrogen sweep was started to remove HCl and excess oxalyl chloride along with tetrahydrofuran. The reactor was then put under a vacuum to remove tetrahydrofaran and isolate the benzoyl chloride product as a pale yellow oil. Final residual solvent was removed under pump vacuum and product was filtered under nitrogen through a coarse fritted glass filter. Near quantitative yield of the benzoyl chloride obtained in this manner can be utilized in the subsequent chemistry without further purification. Samples will crystallize in large crystals if left in the refrigerator but will re-melt at 25° C.

GC retention time of the benzoyl chloride was 7.17 min. Coluin: 30M DB-5 cap column, He @18 psig; 50° C., hold 2 min.,20° C./minute to 250° C. ′H NMR (CDC1₃)δ8.07(m,1 H), 7.25(m, 2H).

### Example 2

Example 2 can be depicted by the following reaction scheme. 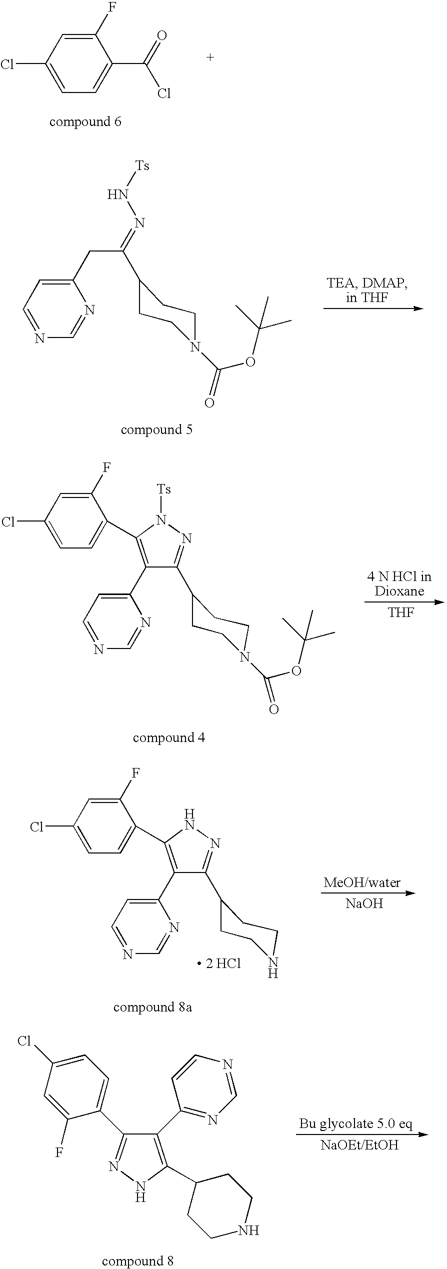 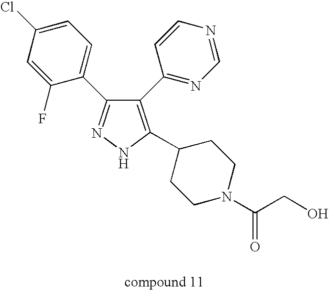
A. Preparation of the Protected Piperidylpyrazole:

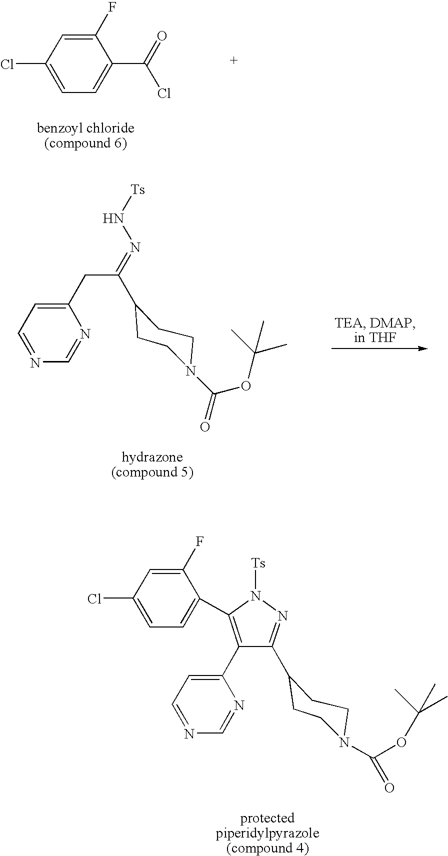

A 1 L addition fuiniel was placed on a 22L round bottom reaction flask fitted with an overhead stirrer. The benzoyl chloride (1100 g, 5.70 moles, 1.44 equivalent) was transferred into a 1 L dropping fumiel. 6L of dry tetrahydrofuran was charged to the reactor and 49 g, (0.40moles, 0.1 equivalent) of 4-dimethylaminopyridine (DMAP) was added to it and stirred until dissolved. The hydrazone (1875 g, 3.96 moles, 1 equivalent) was charged to give a thin slurry. To this stirring slurry was added 675 g (6.68 moles, 1.69 equivalent) of triethylaamine (TEA). The yellow thin slurry was then cooled to under 10° C. and the benzoyl chloride was added in a thin stream over an hour. The addition is added at a rate to keep the batch temperature from rising above 10° C. The batch was allowed to warm after the total amount of benzoyl chloride had been added. The batch was then heated carefully to 50° C. for 30 minutes to finish the reaction. The reaction was cooled to less than 35° C. and filtered to remove triethylamine hydrochloride that had precipitated, usually 700-800 g. The filter cake was washed with 1 L of tetrahydrofuran and the filtrate plus wash was returned to the reactor for subsequent deprotection. The white triethylamine hydrochloride salt was discarded. The product can be utilized without isolation as a solution for the subsequent deprotection reaction to produce the protected piperidylpyrazole. If desired, the protected-piperidylpyrazole can be isolated as a white solid by crystallization using methanol or toluene solvent.

HPLC retention time of the protected piperidylpyrazole (10.75 min.) Column:15 cm Zorbax XDB-C8, ACN/H₂O, gradient 20%-100% @10 min. hold for 10 min. 1.00 mL/min. λ=258 nm. ¹H NMR (CDC1₃) δ9.2 (s, 2H), 8.5 (d, 1H), 7.7 (d, 2H), 7.4-7.1 (m, 4H), 6.8 (d, 1H), 4.1 (m, 2H), 3.3 (s, residual MeOH from crystallization), 3.2 (m, 1H), 2.8 (m, 2H), 2.4 (s, 3H), 1.9-1.6 (m, 5H), 1.4 (s, 9H). Anal. Calc'd for C₃₀H₃₁N₅O₄S₁Cl₁F₁:C, 57.80; H, 5.48; N, 10.87. Found: C, 57.94; H, 5.40; N, 11.05.
B. Preparation of the Unproteted Piperidylpyrazole:

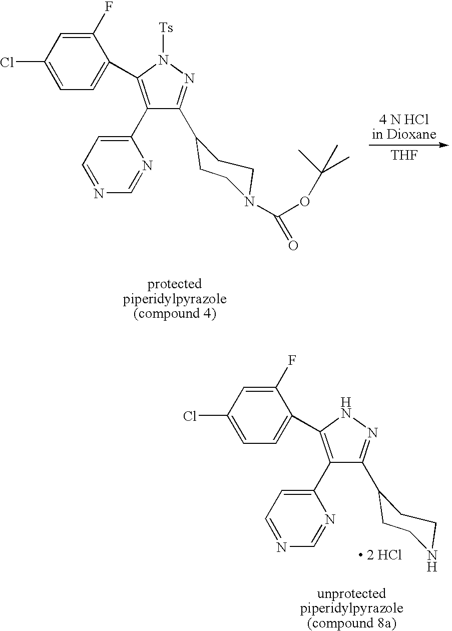

The protected piperidylpyrazole solution described above was charged into a 22 L round bottom reactor together with 2.25 L of tetrahydrofuran. This was followed by 4 L (4 equivalent) of 4 N HCl in dioxane with good stirring. The reaction turned cloudy and slowly formed a clear orange solution. After the batch had stirred for about 10 minutes, another 2 L of 4N HCl in dioxane was added to the batch. The batch was heated to 50° C. for 30 minutes to complete the hydrolysis.

The product was isolated as an aqueous solution for subsequent neutralization by avoiding the filtration step. After the hydrolysis was complete, the solution was cooled to 25° C. and water/toluene in the ratio of 1:2 was added. The resulting solution was mixed for about 0.5 hours and allowed layers to separate upon standing. The organic layer was discarded and the aqueous layer containing the product was washed with toluene to further remove residual organic impurities and utilized in further transformation to prepare a neutral unprotected piperidylpyrazole.

HPLC retention time of the unprotected piperidylpyrazole (4.35 min). Column: 15 cm Zorbax XDB-C8, ACN/H₂O, gradient 20%-100% @10 min. hold for 10 min. 1.00 mL/min. λ=258 nm.
C. Preparation of a Nieutral Unprotected Piperidylpyrazole:

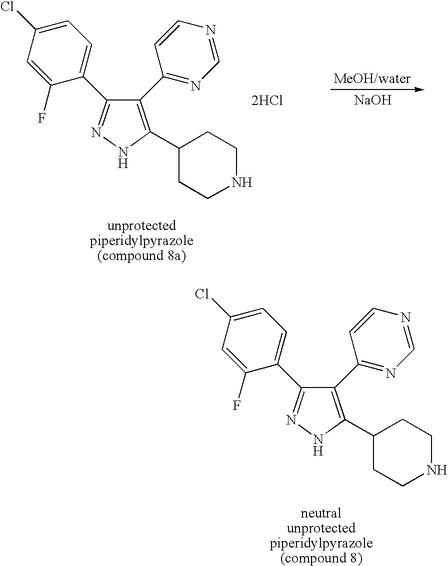

Crude unprotected piperidylpyrazole (100 g, 0.232 mole) was mixed with 300 mL of methanol to form an orange solution. Water (206 mL) was added which resulted in an exotherm to about 33° C. To this solution about 93.8 g of 6N NaOH solution was added and the temperature rose to about 40° C. The neutralization was controlled by pH measurement and additional NaOH can be added to adjust the pH to 10.5-11.5 if desired. The solution turned to a clear dark red brown solution and solids slowly started to crystallize out. The batch was heated and maintained at about 50° C. for about 30 minutes. It was then cooled to 10° C. and the solids were filtered, washed with water (2×200 mL) and acetonitrile (2×200 mL) and dried. 54 g were isolated to give about 70% yield of the neutral unprotected piperidylpyrazole.

¹H NMR (DMSO-d6) δ9.15 (s, 1H), 8.6 (d, 1H), 7.6-7.4 (m 2H), 7.2 (d, 1H 3.0 (m, 3H), 2.5 (m, 3H), 1.8-1.6 (m, 4H). Anal. Calc'd for C₁₈H₁₇N₅Cl₁F₁+0.65%H₂O: C, 58.51; H, 4.99; N, 18.95. Found: C, 58.14; H, 4.63; N, 18.73.
D. Preparation of N-(2-Hydroxyacetyl)-5-(4-Piperidyl)-3-(Phenyl)Pyrazole compound:

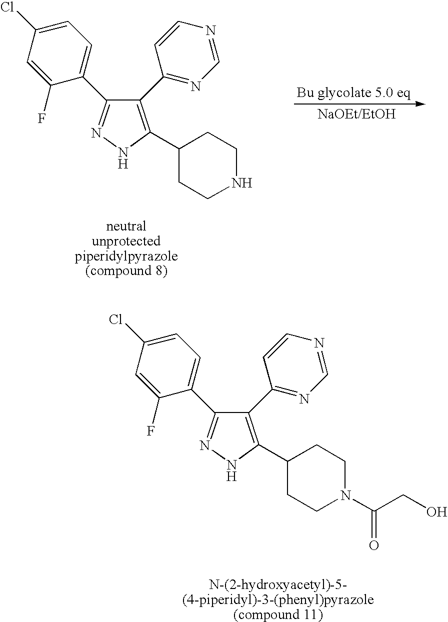

The neutral unprotected piperidylpyrazole (2 kg, 5.59 moles) was mixed with 15 L of absolute ethanol and 3.7 kg (28 moles, 5 equivalent) butyl glycolate at ambient temperature. 20% sodium ethoxide solution (1.8 kg, 1 equivalent) was added to this mixture and the resulting solution was heated to 79-81° C. for a period of 4 hours. Afterwards the solution was cooled to about 5° C. and approximately 2.36 kg of crude product and the corresponding sodium salt were isolated. This crude solid was resuspended in 9.4 L of ethanol and heated to about 40° C. Concentrated HCl (1.3 kg, about 2.4 equivalent) was added via an addition funnel in about 10 minutes and a heat kick was observed. Water (15.7 kg) was then added at such a rate to maintain the pot temperature of 40° C. After about 20% of water added a clear light brown solution was obtained. Afterwards the solution was slowly cooled to 0° C. and the solid filtered, washed four times with 3.8 kg of water and dried to give desired hydrated product (containing about 5% water) in yield of 70-80%.

### Example 3

Example 3 can be depicted by the following reaction scheme. 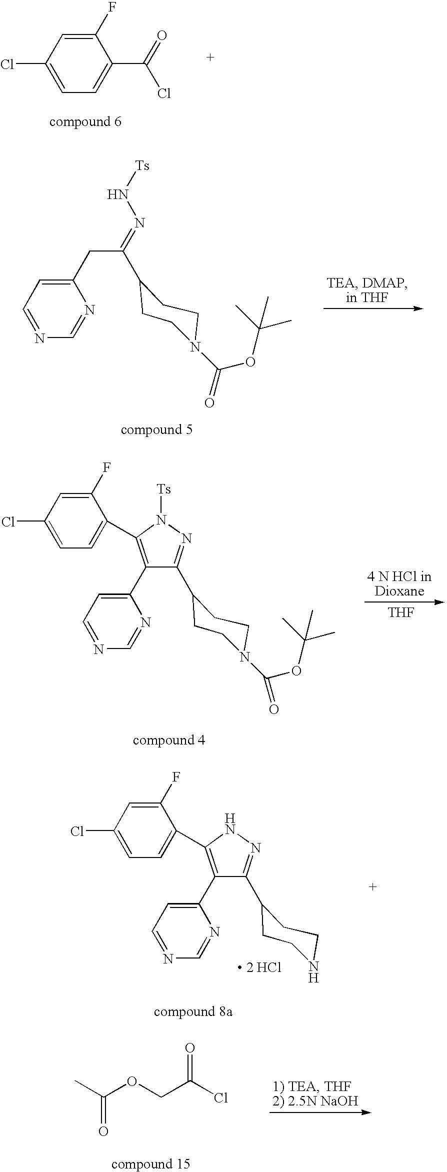 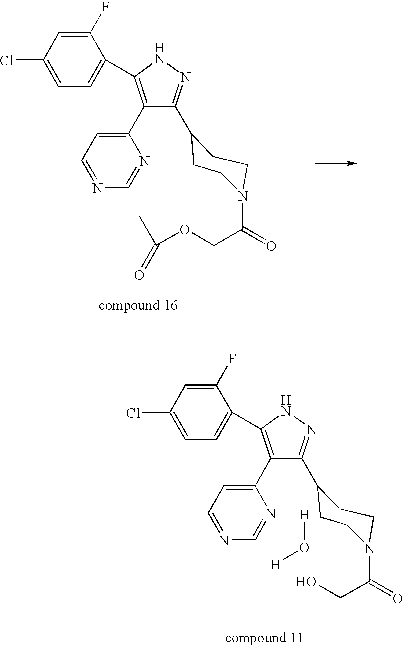
A. Preparation of the Protected Piperidylpyrazole:

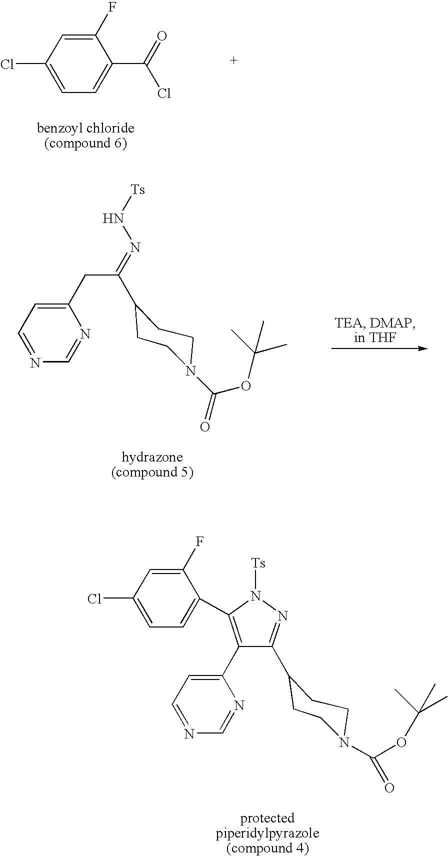

A 1 L addition funnel was placed on a 22 L round bottom reaction flask fitted with an overhead stirrer. The benzoyl chloride (1100 g, 5.70moles, 1.44 equivalent) was transferred into a 1 L dropping funnel. 6 L of dry tetrahydrofuran was charged to the reactor and 49 g, (0.40moles, 0.1 equivalent) of 4-dimethylaminopyridine was added to it and stirred until dissolved. The hydrazone (1875 g, 3.96 moles, 1 equivalent) was charged to give a thin slurry. To this stirring slurry was added 675 g (6.68 moles, 1.69 equivalent) of triethylamine. The yellow thin slurry was then cooled to under 10° C. and the benzoyl chloride was added in a thin stream over an hour. The addition was added at a rate to keep the batch temperature from rising above 10° C. The batch was allowed to warm after the total amount of benzoyl chloride had been added. The batch was then heated carefully to 50° C. for 30 minutes to finish the reaction. The reaction was cooled to less than 35° C. and filtered to remove triethylamine hydrochloride that had precipitated, usually 700-800 g. The filter cake was washed with 1 L of tetrahydrofuran and the filtrate plus wash was returned to the reactor for subsequent deprotection. The white triethylamine hydrochloride salt was discarded. The product can be utilized without isolation as a solution for the subsequent deprotection reaction to produce the unprotected piperidylpyrazole. If desired, the protected piperidylpyrazole can be isolated as a white solid by crystallization using methanol or toluene solvent.

HPLC retention time of the protected piperidylpyrazole (10.75 min.) Column:15 cm Zorbax XDB-C8, ACN/H₂₀, gradient 20%-100% @10 min. hold for 10 min. 1.00 mL/min. λ=258 nm. ¹H NMR (CDCl₃) δ9.2 (s, 2H), 8.5 (d, 1H), 7.7 (d, 2H), 7.4-7.1 (m, 4H), 6.8 (d, 1H), 4.1 (m, 2H), 3.3 (s, residual MeOH from crystallization), 3.2 (m, 1H), 2.8 (m, 2H), 2.4 (s, 3H), 1.9-1.6 (m, 5H), 1.4 (s, 9H). Anal. Calc'd for C₃₀H₃₁N₅O₄S₁Cl₁F₁ : C,57.80;H, 5.48; N, 10.87. Found: C, 57.94;H,5.40; N, 11.05.
B. Preparation of the Unproteted Piperidylpyrazole:

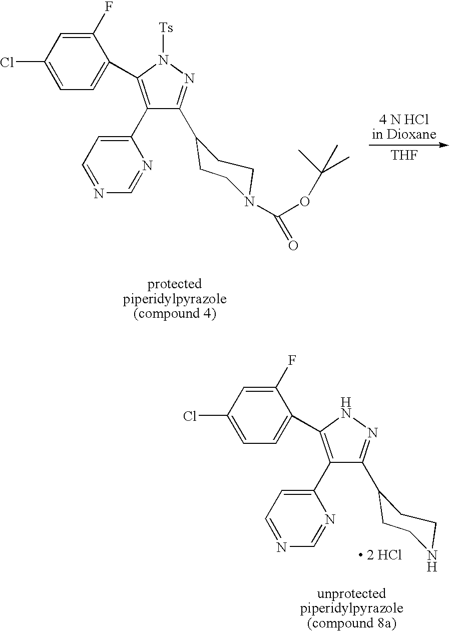

The protected piperidylpyrazole solution described above was charged into a 22 L round bottom reactor together with 2.25 L of tetrahydrofuran. This was followed by 4 L (4 equivalents) of 4 N HCl in dioxane with good stirring. The reaction turned cloudy and slowly formed a clear orange solution. After the batch was stirred for about 10 minutes, another 2 L of 4N HCl in dioxane was added to the batch. The batch was heated to 50° C. for 30 minutes to complete the hydrolysis. The reaction was stirred while solids precipitated out of the solution, giving a fine granular powder. After stirring for several hours at room temperature, the batch was filtered to isolate the hydrochloride salt and the filter cake was given two washes with 2.5 L of tetrahydrofuran. The solid was dried on the filter under a stream of nitrogen. Total isolated yield was 1790 g. The solid usually contains some 10-11% of triethylamine hydrochloride but does not interfere in the next step.

HPLC retention time of the unprotected piperidylpyrazole (4.35 min). Colurn: 15 cm Zorbax XDB-C8, ACN/H₂O, gradient 20%-100% @10 min. hold for 10 min. 1.00 mL/min. λ=258 nm.
C. Preparation of N-(2-Hydroxyacetyl)-5-(4-Piperidyl)-3-(Phenyl)Pyrazole:

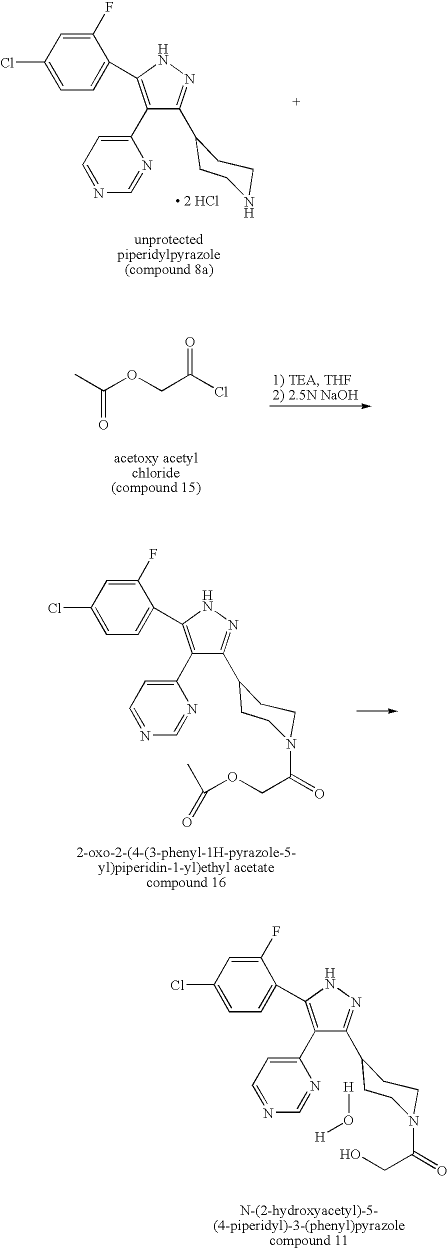

A 12 L round bottom flask fitted with overhead stirrer, 1 L addition funnel, and reflux condenser was charged with 2.75 L of tetrahydrofuran. The unprotected piperidylpyrazole (484 g, est.1.123 moles, 1 equivalent) was slurried in and cooled to about 0° C. Triethylamine (606 g, 5.989 moles, 5.34 equivalent) was slowly added to the batch and 247 g(1.809 moles, 1.61 equivalent ) of acetoxyacetyl chloride was added dropwise keeping the temperature at about 0° C. to 5° C. over about 1 hour period. The reaction was followed by LC analysis. It was then heated to 50° C. for 30 minutes and then cooled back to 25° C. and immediately filtered free of triethylamine HCl salt that had precipitated. The filter cake was washed twice with 500 mL tetrahydrofuran and discarded. The filtrate and the washes were returned to the reactor and treated with 770 mL of methanol. The batch was cooled to 0° C. and 310 mL of 2.5 N NaOH solution was added, keeping the batch temperature under 10° C. An LC sample verified that the hydrolysis to N-(2-hydroxyacetyl)-5-(4-piperidyl)-3-(phenyl)pyrazole was complete. Then 76 g of concentrated HCI diluted with 1850 mL deionized water was added. The reaction was concentrated in vacuo and the product precipitated out from the aqueous media. The product solids were filtered and washed with twice with 1 L water and 600 mL acetone, and dried. A total of 296 g of product N-(2-hydroxyacetyl)-5-(4-piperidyl)-3-(phenyl)pyrazole was isolated.

HPLC retention time of N-(2-hydroxyacetyl)-5-(4-piperidyl)-3-(phenyl)pyrazole (5.60 min). Column: 15 cm Zorbax XDB-C8, ACN/H₂0, gradient 20%-100% @10 min. hold for 10 min. 1.00 mL/min. λ=258 nm. ¹H NMR (dmso-d6): δ13.4 (s, 1H), 9.18 (s, 1H), 8.65 (d, 1H), 7.6-7.2 (m, 3H), 7.18 (d, 2), 4.6-4.4(m, 2H), 4.2 (m 2H), 3.9-3.4 (m, 2H), 3.1 (m, 1H), 2.8 (m, 1H), 2.0-1.6 (m, 4H). Anal. Calc'd for C₂₀H₁₉N₅O₂Cl₁F₁+1.4% H₂O: C, 54.46; H, 4.98; N, 15.88. Found: C, 54.87; H, 5.02; N, 15.87.

The examples herein can be performed by substituting the generically or specifically described reactants and/or operating conditions of this invention for those used in the preceding examples.

In view of the above, it will be seen that the several objects of the invention are achieved. As various changes could be made in the above methods, combinations and compositions of the present invention without departing from the scope of the invention, it is intended that all matter contained in the above description be interpreted as illustrative and not in a limiting sense. All documents mentioned in this application are expressly incorporated by reference as if fully set forth at length.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

1. A crystalline form of 2-{4-[3-(4-chloro-2-fluorophenyl)-4-pyrimidin-4-yl-1H-pyrazol-5-yl]piperidin-1-yl}-2-oxoethanol.

2. A crystalline form of claim 1 having an X-ray powder diffraction pattern comprising a peak selected from the group consisting of 8.3±0.2, 11.7 ±0.2, 16.7±0.2, 21.2±0.2, 24.8±0.2, 27.7±0.2 , and 28.5±0.2degrees 2 theta

3. A crystalline form of claim 1 having a melting point in a range from about 213° C. to about 217° C.

4. A crystalline form of claim 1 having an infrared absorption band profile comprising an absorption band at about 1644 cm⁻¹.

5. A crystalline form of claim 1 having a melting point in a range from about 213° C. to about 217° C., an infrared absorption band profile comprising an absorption band at about 1644 cm⁻¹, and an X-ray powder diffraction pattern comprising peaks at 11.7±0.2 and 28.5±0.2 degrees 2 theta.

6. A crystalline form of claim 1 having an X-ray powder diffraction pattern substantially as shown in FIG. 1.

7. A pharmaceutical composition comprising 2-{4-[3-{4-chloro-2-fluorophenyl)-4-pyrimidin-4-yl-]1H-pyrazol-5-yl]piperidin-1-yl}-2-oxoethanol and one or more pharmaceutically acceptable excipients, wherein a detectable amount of said 2-{4-[3-(4-chloro-2-fluorophenyl)-4-pyrimidin-4-yl-1H-pyrazol-5-yl]piperidin-1-yl}-2-oxoethanol is present as Form 1 crystalline 2-{4-[3-(4-chloro-2-fluorophenyl)-4-pyrimidin-4-yl-1H-pyrazol-5-yl]piperidin-1-yl}-2-oxoethanol, wherein Form 1 has a melting point in a range from about 213° C. to about 217° C. an infrared absorption band profile comprising an absorption band at about 1644 cm⁻¹, and an X-ray powder diffraction pattern comprising peaks at 11.7±0.2 and 28.5±0.2 degrees 2 theta.

8. The pharmaceutical composition of claim 7 wherein at least about 50% of said 2-{4-[3-(4-chloro-2-fluorophenyl)-4-pyrimidin-4-yl-1H-pyrazol-5-yl]piperidin-1-yl}-2-oxoethanol is present as Form 1 crystalline 2-{4-[3-(4-chloro-2-fluorophenyl)-4-pyrimidin-4-yl-1H-pyrazol-5-yl]piperidin-1-yl}-2-oxoethanol.

9. The pharmaceutical composition of claim 8 wherein at least about 90% of said 2-{4-[3-(4-chloro-2-fluorophenyl)-4-pyrimidin-4-yl-1H-pyrazol-5-yl]piperidin-1-yl}-2-oxoethanol is present as Form 1 crystalline 2-{4-[3-(4-chloro-2-fluorophenyl)-4-pyrimidin-4-yl-1H-pyrazol-5-yl}piperidin-1-yl}-2-oxoethanol.

10. The pharmaceutical composition of claim 9 wherein said 2-{4-[3-(4-chloro-2-fluorophenyl)-4-pyrimidin-4-yl-1H-pyrazol-5-yl]piperidin-1-yl}-2-oxoethanol present in the composition is substantially phase pure Form 1 crystalline 2-{4-[3-(4-chloro-2-fluorophenyl)-4-pyrimidin-4-yl-1H-pyrazol-5-yl]piperidin-1-yl}-2-oxoethanol.

11. The pharmaceutical composition of claim 7 wherein the amount of said 2-{4-[3-(4-chloro-2-fluorophenyl)-4-pyrimidin-4-yl-1H-pyrazol-5-yl]piperidin -1-yl}-2-oxoethanol present in the composition is between about 0.1 mg to about 1000 mg.

12. The pharmaceutical composition of claim 11 wherein the amount of said 2-{4-[3-(4-chloro-2-fluorophenyl)-4-pyrimidin-4-yl-1H-pyrazol-5-yl]piperidin -1-yl)2-oxoethanol present in the composition is between about 0.1 mg to about 500 mg.

13. A method of treating or preventing a p38 kinase-mediated condition, the method comprising administering to a subject having or susceptible to such condition or disorder a therapeutically or prophylactically effective amount of the composition of claim 7.

14. The method of claim 13 wherein the p38 kinase-mediated condition is rheumatoid arthritis.
